(19) United States
(12) Patent Application Publication
(10) Pub. No.: US 2009/0095308 A1
(43) Pub. Date: Apr. 16, 2009

(54) Support device

(76) Inventor: Wade Mckinney, (US)

Correspondence Address: Wade McKinney, 5 Splawn Court, Canyon, TX, 79015, US

(21) Appl. No.: 11/974,767

(22) Filed: Oct. 16, 2007

(51) Int. Cl. A61F 5/37; A61F 5/00

(52) U.S. Cl. 128/870; 602/19

(57) ABSTRACT

A support device comprising a first arch for a left-side anterior torso having a first superior contact point and a first inferior contact point; a second arch for a right-side anterior torso having a second superior contact point and a second inferior contact point; an anterior connecting means to connect said first longitudinal extended arch with said second longitudinal extended arch; and a posterior connecting means to connect said first longitudinal extended arch with said second longitudinal extended arch; whereby said extended portions restrict movement of a spine during a motion, and associated method of use.

### TECHNICAL FIELD

The presently preferred embodiment of the innovations described herein relate generally to providing support, and more specifically, to a device that restricts the use of the spine in a flexion.

### BACKGROUND

Preventing back injuries is a major workplace safety challenge. According to the United States Bureau of Labor Statistics (BLS), more than one million workers suffer back injuries each year, and back injuries account for one of every five workplace injuries or illnesses. Further, one-fourth of all compensation indemnity claims involve back injuries, costing industry billions of dollars on top of the pain and suffering borne by employees.

Moreover, though lifting, placing, carrying, holding and lowering are involved in manual materials handling (the principal cause of compensable work injuries) the BLS survey shows that four out of five of these injuries were to the lower back because of inadequate lower back support, and that three out of four occurred while the employee was lifting.

It goes without saying that maintaining a straight back is an important element to personal safety when lifting heavy objects. Even the United States Government has a Back and Lifting Training Guide, an example of which is located at: www.cdc.gov/elcosh/docs/d0200/d000253/d000253.pdf that highlights proper techniques for lifting heavy objects.

However, no approach has been found for totally eliminating back injuries caused by lifting—even the commonly seen lifting belts that allegedly support the lower back. But the drawback with lower back support belts and training, is the human error factor where an individual might still bend forward at the waist while their lower back is supported through the extension of the back. One approach to help reduce back injuries has been through improved ergonomic programs proposed by the United States Department of Labor, Occupational Safety and Health Administration.

What is needed is a support device that encourages proper ergonomic lifting techniques to limit the movement of the spine during the flexion of the back.

### SUMMARY

To achieve the foregoing, and in accordance with the purpose of the presently preferred embodiment as broadly described herein, the present application provides a support device comprising a first arch for a left-side anterior torso having a first superior contact point and a first inferior contact point; a second arch for a right-side anterior torso having a second superior contact point and a second inferior contact point; an anterior connecting means to connect said first longitudinal extended arch with said second longitudinal extended arch; and a posterior connecting means to connect said first longitudinal extended arch with said second longitudinal extended arch; whereby said extended portions restrict movement of a spine during a motion. The device, wherein said first arch is a longitudinal extended arch. The device, wherein said second arch is a longitudinal extended arch. The device, wherein said first superior contact point and said second superior contact point is a sternum. The device, wherein said first inferior contact point and said second inferior contact point is a pelvis. The device, wherein said spine is a lumbar region of a vertebral column. The device, wherein said movement is the flexion movement of the spine.

Another advantage of the presently preferred embodiment is to provide a method for lifting objects while restricting movement of a spine, comprising attaching a support device to a human torso; bending at the knees to grasp an object; moving said object while wearing said support device; and reinforcing safe body mechanics through use of the support device while moving said object such that said support device prevents the flexion of the lumbar spine.

And another advantage of the presently preferred embodiment is to provide a support device comprising a first longitudinal extended arch for a left-side anterior torso having a first sternum contact point and a first pelvis contact point; a second longitudinal extended arch for a right-side anterior torso having a second sternum contact point and a second pelvis contact point; an anterior connecting means to connect said first longitudinal extended arch with said second longitudinal extended arch; and a posterior connecting means to connect said first longitudinal extended arch with said second longitudinal extended arch; whereby said extended portions restrict the flexion of a lumbar spine.

Other advantages of the presently preferred embodiment will be set forth in part in the description and in the drawings that follow, and, in part will be learned by practice of the presently preferred embodiment. The presently preferred embodiment will now be described with reference made to the following Figures that form a part hereof. It is understood that other embodiments may be utilized and changes may be made without departing from the scope of the presently preferred embodiment.

### BRIEF DESCRIPTION OF THE DRAWINGS

A presently preferred embodiment will hereinafter be described in conjunction with the appended drawings, wherein like designations denote like elements, and:

FIG. 1 is an anterior view of a support device when worn by a person;

FIG. 2 is a posterior view of a support device when worn by a person; and

FIGS. 3a & FIG. 3b illustrate the first arch with a first superior section separated From a first inferior section.

### DETAILED DESCRIPTION OF THE PREFERRED EMBODIMENTS

The numerous innovative teachings of the present application will be described with particular reference to the presently preferred embodiments. It should be understood, however, that this class of embodiments provides only a few examples of the many advantageous uses of the innovative teachings herein. The presently preferred embodiment provides, among other things, a device for supported lifting, and related method.

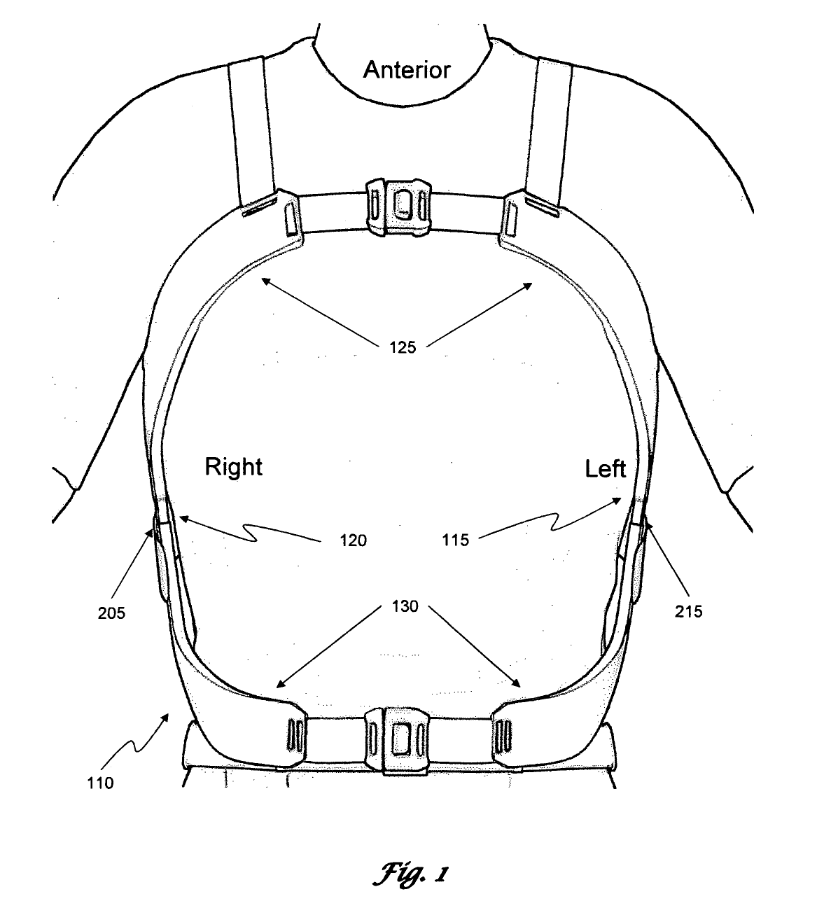

FIG. 1 is an anterior view of the support device on a torso. Referring now to FIG. 1, a support device 110 worn on a torso of a person to limit the movement of the person's spine during flexion of the back, where the flexion/extension motion of the back can be used for lifting or placing, for example. The spine is a common term for the vertebral column that comprises different regions, where those regions are called cervical, thoracic, lumbar, and pelvic. The support device 110 has a first arch 115 extending longitudinally along a left anterior portion of the torso and a second arch 120 extending longitudinally along a right anterior portion of the torso. Both arches 115,120 are preferable constructed of hardened plastic formed by stamping or injection molding technologies. Alternatively, the arches 115,120 can be formed from steel, metal, or other matter. Regardless, the material of the arches 115,120 is of sufficient strength so as to not fail during the flexion of the back. The first arch 115 and the second arch 120 are conformed to generally contact the person at a superior contact point 125 located at or near the person's sternum. And the first arch 115 and the second arch 120 are conformed to generally contact the person at an inferior contact point 130 at or near the person's pelvis. The first arch 115 and the second arch 120 connect to each other across the torso using a dual anterior belt system at two belt locations. The first belt location is at the superior contact point 125, and the second belt location is at the inferior contact point 130.

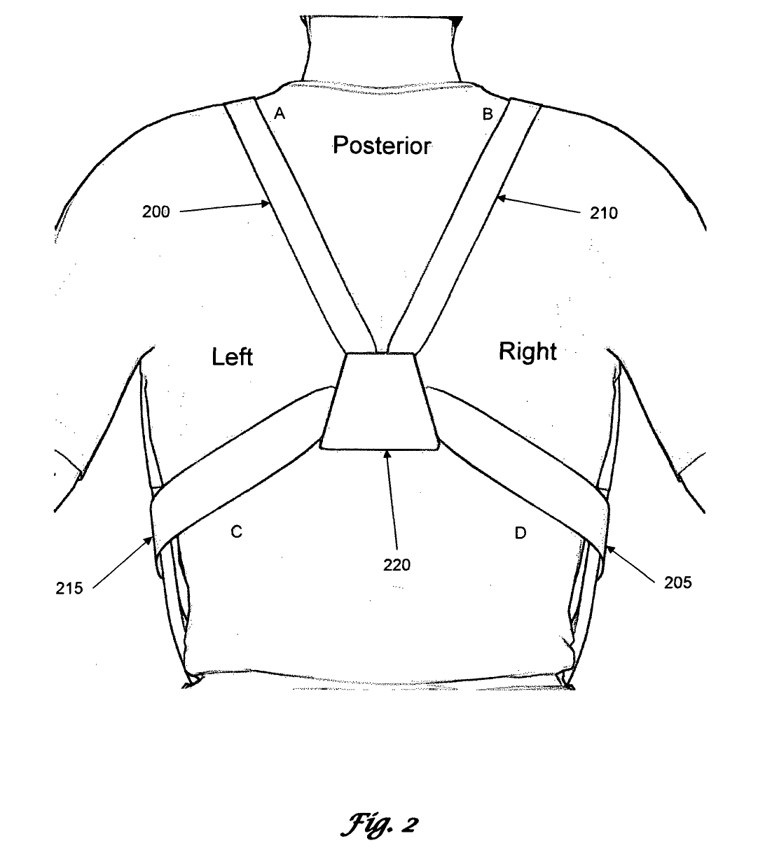

When viewed from the posterior, as illustrated in FIG. 2, the first arch 115 and the second arch 120 connect to each other using a dual posterior belt system in an X-like fashion. A first posterior belt 200 (indicated by belt labels A & D) connects to the first arch 115 at the superior contact point 125 and crosses the person's back to attach to a midpoint of the second arch, illustrated at 205. A second posterior belt 210 (identified by belt labels B & C) connects to the second arch 120 at the superior contact point 125 and crosses the person's back to attach to a midpoint of the first arch, illustrated at 215. A back-strap accumulator 220 helps arrange and coordinate the belt labels A-D. In an alternative embodiment, there are four individual belts that connect into the back-strap accumulator 220. Alternatively, there can be three belt system for securing the support device 110 to the person's back. Likewise it is appreciated that any method to secure the support device 110 to the person's torso is contemplated such that the superior and inferior contact points 125,130, respectively, are properly engaged.

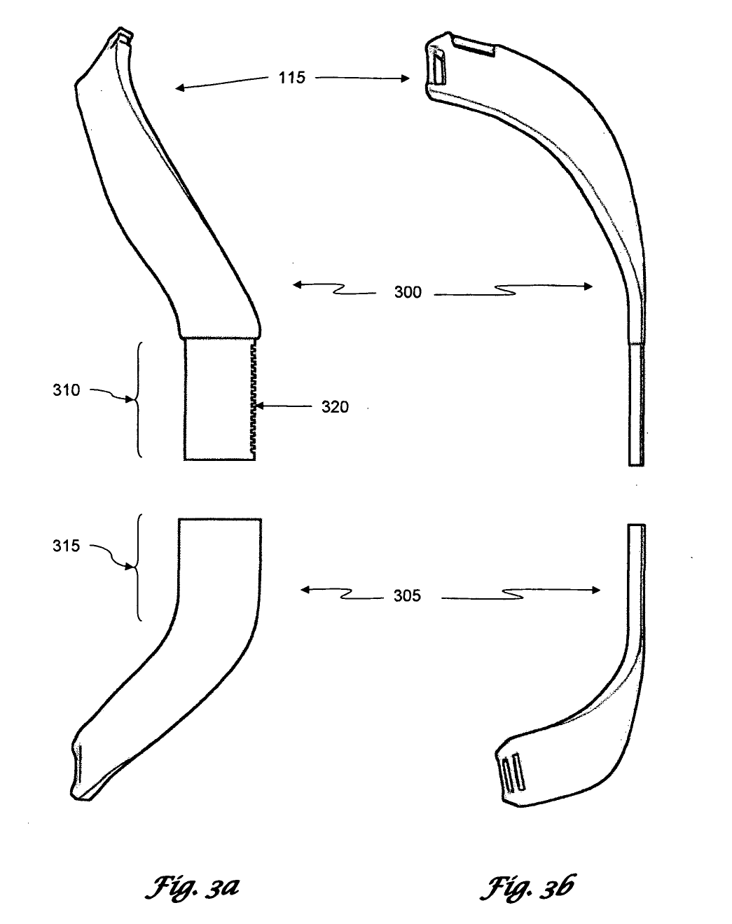

FIGS. 3a & 3b illustrate the first arch 115 with a first superior section 300 separated from a first inferior section 305. Further, the first arch 115 and second arch 120 are mirror images of one another, and therefore the following discussion of the first arch 115 applies to the second arch 120, and vice versa. A lower rigid member 310 of the first superior section 300 connects to an upper rigid member 315 of the first inferior section 4305. Alternatively, the rigid members 310, 315 connect to one another preferably by use of a connecting member having a number of screw-holes whereby each rigid member is attached to the connecting member using a nut/bolt combination or other commonly acceptable screw-attachment mechanism. In an alternate embodiment, the upper rigid member 3415 has a curved surface with vertical flanges extended therefrom that slidingly engages the lower rigid member 4310 such that the rigid members 310,315 are then securely connected by a nut/bolt, for example. One skilled in the art can appreciate that other means are available to connect the two arches, such as that seen in FIG. 3a where the lower rigid member 310 has teeth 320 fitted to engage with matting teeth (not depicted) on the upper rigid member 315, whereby the two sections 300,305 securely connect to each another.

In application, the user places the support device 110 on the torso. With the intention of moving an object from a first location to a second location, the user grasps the object and is restricted to using bended knees to move the object in the vertical direction because the user feels restricted to “bend forward from the waist” or the flexion of the spine based on the placement of the support device. By having restricted flexion of the spine, the support device also promotes ergonomic lifting of heavy objects without the need to focus on a lower back supporting apparatus.

A number of embodiments have been described. It will be understood that various modifications may be made without departing from the spirit and scope of the presently preferred embodiment, such as the addition of a sensor for detecting excessive force/strain in flexion in torque or forward angle where the sensor signals to the user by auditor means when excessive force/strain is detected. Likewise, a recording means can be employed to store data related to users of the support device that are likely to be subject to back injuries, or record position data of the support device for use in injury claims. Therefore, other implementations are within the scope of the following claims.

1. A support device comprising:
a first arch for a left-side anterior torso having a first superior contact point and a first inferior contact point;
a second arch for a right-side anterior torso having a second superior contact point and a second inferior contact point;
an anterior connecting means to connect said first longitudinal extended arch with said second longitudinal extended arch; and
a posterior connecting means to connect said first longitudinal extended arch with said second longitudinal extended arch;
whereby said extended portions restrict movement of a spine during a motion.

2. The device of claim 1, wherein said first arch is a longitudinal extended arch.

3. The device of claim 1, wherein said second arch is a longitudinal extended arch.

4. The device of claim 1, wherein said first superior contact point and said second superior contact point is a sternum.

5. The device of claim 1, wherein said first inferior contact point and said second inferior contact point is a pelvis.

6. The device of claim 1, wherein said spine is a lumbar region of a vertebral column.

7. The device of claim 1, wherein said movement is the flexion movement of the spine.

8. A method for lifting objects while restricting movement of a spine, comprising:
attaching a support device to a human torso;
bending at the knees to grasp an object;
moving said object while wearing said support device; and
reinforcing safe body mechanics through use of the support device while moving said object such that said support device prevents the flexion of the lumbar spine.

9. A support device comprising:
a first longitudinal extended arch for a left-side anterior torso having a first sternum contact point and a first pelvis contact point;
a second longitudinal extended arch for a right-side anterior torso having a second sternum contact point and a second pelvis contact point;
an anterior connecting means to connect said first longitudinal extended arch with said second longitudinal extended arch; and
a posterior connecting means to connect said first longitudinal extended arch with said second longitudinal extended arch;
whereby said extended portions restrict the flexion of a lumbar spine.
